(19) United States
(12) Patent Application Publication
(10) Pub. No.: US 2026/0224765 A1
(43) Pub. Date: Aug. 6, 2026

(54) WATER AND AIR DISINFECTION INTEGRATED IN A NITI BASED SYSTEM

(71) Applicant: EXERGYN LIMITED, Dublin (IE)

(72) Inventors: Michael LANGAN, Dublin 11 (IE); Philip SHEPPARD, Dublin 11 (IE)

(73) Assignee: EXERGYN LIMITED, Dublin (IE)

(21) Appl. No.: 18/869,002

(22) PCT Filed: May 29, 2023
(86) PCT No.: PCT/EP2023/064340

§ 371 Date: Nov. 25, 2024

(30) Foreign Application Priority Data
May 27, 2022 (GB) 2207843.0

(51) Int. Cl. A61L 2/088; A61L 2/104; A61L 9/20; A61L 103/15; B01J 23/755; B01J 35/39; C02F 1/32; C02F 1/72; F24F 8/22

(52) U.S. Cl. CPC A61L 2/088; A61L 2/104; A61L 9/205; B01J 23/755; B01J 35/39; C02F 1/325; C02F 1/725; F24F 8/22; A61L 2103/15; A61L 2202/11; A61L 2202/14; A61L 2209/111; A61L 2209/12; C02F 2305/023; C02F 2305/10

(57) ABSTRACT

Disclosed is a disinfection system that includes a NiTi based component, wherein an exposed surface of the NiTi based component includes titanium atoms in oxidised form as TiO₂, and a UVC source for projecting UVC light onto the exposed surface of the NiTi based component to react with the titanium oxide present at the exposed surface, wherein the exposure of the TiO₂ with UVC light creates radicals that destroy organic matter present between the UVC source and the exposed surface of the NiTi based component.

### FIELD

The present disclosure is directed towards water and air disinfection/sterilisation, and more particularly to enhanced disinfection of heat transfer fluids integrated into heat pump cycles.

### BACKGROUND

Heat pumps/air conditioning (AC) are used in Heating, Ventilation, Air Conditioning and Refrigeration (HVAC-R) systems. Such systems are subjected to regulations to ensure safety from waterborne pathogens such as legionella and can reduce rates of infection from airborne pathogens, such as coronavirus.

The use of UV, and more particularly UVC, for disinfection of water and air is known and established. The UVC alone is proven to denature biological material and is used in existing disinfection products. The efficacy of the disinfection can be enhanced significantly if the UVC irradiation is combined with photocatalysts. The use of Titanium Dioxide (TiO₂) as a photocatalyst in conjunction with UVC is also known and established. Titanium is highly reactive and oxidises rapidly to form TiO₂ at the surface. A disinfection system with both UVC and photocatalyst is more compact and uses less power than UVC alone.

Existing HVAC systems include heat pumps serving hydronic heat emitters or chillers in which a single fluid temperature input is used, and a series of valves is used at the output of the chamber to direct the colder fluid flow from the heat absorption phase back to source, while directing the warmer fluid from the heat release phase to the heating target. A common control for contamination of the fluid in such heat pump systems is an additional resistance or other type of heater to take the water above 60° C., which destroys bacteria such as Legionella.

Existing HVAC systems also include air conditioning heat pumps providing temperature-controlled air circulation. Some such systems also enhance air quality using additional components such as membrane filters and UV treatment, and are now particularly aimed at removing viruses such as Covid 19.

In both heat pump applications, circulating fluid, control of contamination, for example caused by biological pathogens and the like, is achieved with equipment which is additional to the equipment which delivers the main heating or cooling function. This increases overall operating costs, maintenance times, space required and weight of such systems.

CN 111397018 discloses an air purification system in which a memory alloy is used to widen an annulus so the volume of air exposed to UV light and a titanium coating on a cloth is increased. The disinfection function is provided by direct UV irradiation and by the radicals formed by exposure of a titanium dioxide coating on the cloth. This is an example of an additional assembly which could be added to the above designs of heat pump, incurring the penalties such as increased overall operating costs, maintenance times, space required and weight of such systems. Therefore, there is a need for to reduce the penalties in heat pumps and in other applications, in which shape memory alloys are used to provide a function unrelated to air or water quality.

Memory alloy is further used in reusable medical devices, of which surgical instruments such as some types of forceps, retractors and scissors are examples. These require both cleaning (removal of organic material) and sterilisation as the highest level of disinfection. These functions are currently fulfilled by a pre-cleaning stage, in which any visible contaminating material is removed and can often involve soaking instruments in water or an enzymatic detergent solution, and a decontamination stage to eliminate microorganisms, which is typically achieved through a combination of manual cleaning and automated equipment. These stages involve costs in the form of time taken to perform and the energy and water used by equipment.

Hence, in view of the above, there is a need for a disinfectant method and system that overcomes the disadvantages of the existing systems.

### SUMMARY

According to the invention there is provided, as set out in the appended claims, a disinfection system that includes a Shape Memory Alloy (SMA) element having a NiTi based component, wherein an exposed surface of the NiTi based component includes titanium atoms in oxidised form as TiO₂, and a UVC source for projecting UVC light onto the exposed surface of the NiTi based component to react with the TiO₂ present at the exposed surface, wherein the exposure of the TiO₂ with UVC light creates radicals that destroy or damage organic matter present between the UVC source and the exposed surface of the NiTi based component.

In an embodiment of the present invention, there is provided a NiTi based component that has an exposed surface including titanium atoms in oxidised form as TiO₂, and a UVC source for projecting UVC light onto the exposed surface of the NiTi based component to react with the TiO₂ present at the exposed surface, wherein the exposure of the TiO₂ with UVC light creates radicals that denature or break down organic matter present in a fluid between the UVC source and the exposed surface of the NiTi based component.

In embodiments of the present invention, the NiTi based component is a Shape Memory Alloy (SMA) core of a heat pump system that performs respectively cooling and/or heating of fluids and the UVC source projects UVC light onto the SMA core for sterilising water or air flowing across the core.

In an embodiment of the present invention, the SMA core is designed to increase the reflection of UVC around the exposed surface.

In an embodiment of the present invention, the cycling between pressurised and depressurised state in the heat pump system may increase the rate of pathogen destruction during water disinfection.

In an embodiment of the present invention, the system further includes sensors in the heat pump system for measuring flow rate, width of flow channel, and fluid turbulence to enhance the efficiency of water or air disinfection.

In an embodiment of the present invention, the water or air disinfection prevents build-up of biofilm on the exposed surface of the SMA core.

In an embodiment of the present invention, when the medium to be disinfected is water, the system further comprises a transparent barrier between the UVC source and the NiTi based component to prevent contact of water with the UVC source.

In an embodiment of the present invention, the NiTi based component is a part of a medical device.

In an embodiment the concentration of Ti at the surface is increased and the concentration of Ni is decreased by treatment of the material e.g. laser processing. This increases the density of active photocatalytic sites.

In another aspect of the present invention, there is provided a heat pump system that includes a NiTi based core positioned in a housing and adapted to undergo stress in response to a first fluid inputted at a first temperature, wherein the NiTi based core comprises a plurality of elements arranged to define the core; and a UVC source positioned at a pre-defined distance from the housing for projecting UVC light towards an exposed surface of the NiTi based core to react with titanium oxide (TiO₂) present on the exposed surface of the NiTi based core, wherein the exposure of the TiO₂ with UVC light creates radicals that disinfect fluid in the housing. The heat pump further comprises a transparent barrier between the UVC source and the NiTi based core to prevent contact of water with the UVC source, when the fluid is water.

In another aspect of the present invention, there is provided a disinfection method that includes providing an exposed surface of the NiTi based component that includes titanium atoms in oxidised form as TiO₂, and projecting UVC light onto the exposed surface of the NiTi based component to react with the TiO₂ present at the exposed surface, wherein the exposure of the TiO₂ with UVC light creates radicals that denature or break down organic matter present in a fluid between the UVC source and the exposed surface of the NiTi based component.

The advantages of the claimed invention are that control of contamination, for example caused by biological pathogens and the like, is achieved partly as an integral part of the system with reduced additional equipment and is achieved more completely. This then allows fluid flow rates to be increased above those possible in systems relying on UVC alone, providing advantages for SMA heat pumps as described below.

The integration of water disinfection/sterilisation technology into the heat pump system that has a NiTi based core reduces the risk of contagion from hazardous biological matter, e.g. legionella, by providing both a non-temperature-based control mechanism in addition to the existing temperature controls and TiO₂-catalysed radicals in addition to the sterilising effect of UVC directly. It increases the likelihood of micro-organisms which survive temperature controls, which may or may not be in place or operating to the designed standard, being destroyed or damaged. Similarly, the integration of the invention into air conditioning systems prevents contagion by destroying airborne pathogens such as coronaviruses. In both cases, the function added to the SMA through the reactivity of TiO₂ reduces the additional equipment required compared to the prior art, so lowering operating costs for end users, reducing maintenance times and enabling easier compliance. Herein, no additional assembly of SMA alloy is required for disinfection purposes, as the SMA alloy used in heat pump performs an additional function of disinfection, thereby reducing overall operating costs, maintenance times, required space and overall weight of system.

Also, the water disinfection/sterilisation prevents the build-up of biofilm on the NiTi based heat transfer surface in the heat pump system and so increases reliability and efficiency. It will be appreciated that biofilm in water provides a suitable environment for pathogen breeding while radicals can reduce biofilm build up elsewhere in hydronic HVAC systems. The TiO₂ substrate is part of the existing NiTi-based heat pump system, so no additional costs are incurred in creating the photocatalyst surface, and no additional space is required. The UV source is the main additional cost, which is a small cost increase over standard systems, but with premium output.

Further, the disinfection method of the present invention can be implemented in those reusable medical devices that include SMA alloy. This reduces the need of manual cleaning and reduces costs in the form of time taken to perform and the energy and water used by equipment.

### BRIEF DESCRIPTION OF THE DRAWINGS

The invention will be more clearly understood from the following description of an embodiment thereof, given by way of example only, with reference to the accompanying drawings, in which:

FIG. 1 illustrates a water disinfection system for a hydronic system, in accordance with an embodiment of the present invention;

FIG. 2 illustrates an air disinfection system, in accordance with an embodiment of the present invention;

FIG. 3 illustrates integration of the UVC sterilization system of FIG. 1 in a conventional water-cooled vapour compression chiller, in accordance with an embodiment of the present invention;

FIG. 4A illustrates a water-cooled SMA chiller with integrated UV sterilization, in accordance with an embodiment of the present invention; and

FIG. 4B illustrates an enlarged view of an SMA core of the water-cooled SMA chiller of FIG. 4A, in accordance with an embodiment of the present invention.

### DETAILED DESCRIPTION OF THE DRAWINGS

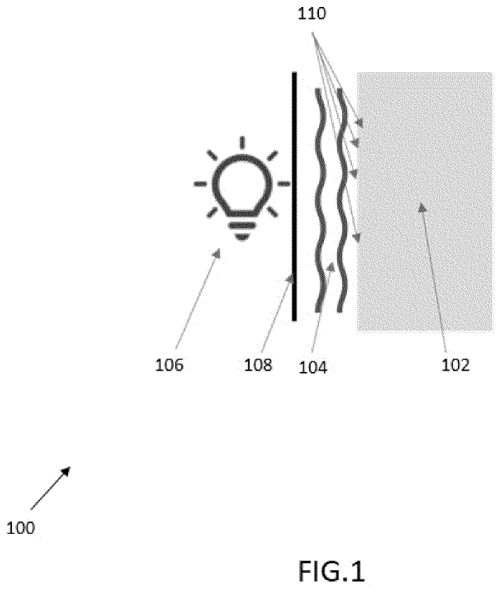

FIG. 1 illustrates a water disinfection system 100 for a hydronic system, in accordance with an embodiment of the present invention.

The water disinfection system 100 includes an NiTi based component 102, working fluid 104 in contact with the top surface of the NiTi based component 102, a UVC source 106 for projecting UVC light towards the working fluid 104 and the NiTi based component 102, and a transparent barrier 108 between the UVC source 106 and the working fluid 104 to prevent contact of the working fluid 104 with the UVC source 106. The top surface of the NiTi based component 102 includes titanium atoms in oxidised form as TiO₂ 110.

An example of the working fluid 104 is water, an example of the UVC source 106 is a UVC lamp, and an example of the transparent barrier 108 is a transparent pipe. In another example the transparent barrier 108 is positioned along one side of the housing.

The exposure of the TiO₂ 110 to UV light of wavelength UVC from the UV lamp 102 creates radicals in the layer of the working fluid 104 near the top surface of the NiTi based component 102. The TiO₂ 110 active sites catalyse the formation of radicals which are highly reactive. These radicals are highly reactive, short-lived and significantly increase the efficacy of UV disinfection. They react with organic matter within a short distance of the surface, are rapidly denatured, do not contaminate the working fluid as they are short-lived and so do not contaminate the working fluid, and are fully consumed before exiting the reaction chamber.

It will be appreciated that in the context of the present invention the terms disinfection/sterilisation/cleaning are to be used broadly in the context of the function of the UVC light source. The UVC light source is configured to generate UVC light sufficient to create radicals that denature or break down organic matter present between the UVC source and the exposed surface of the NiTi based component.

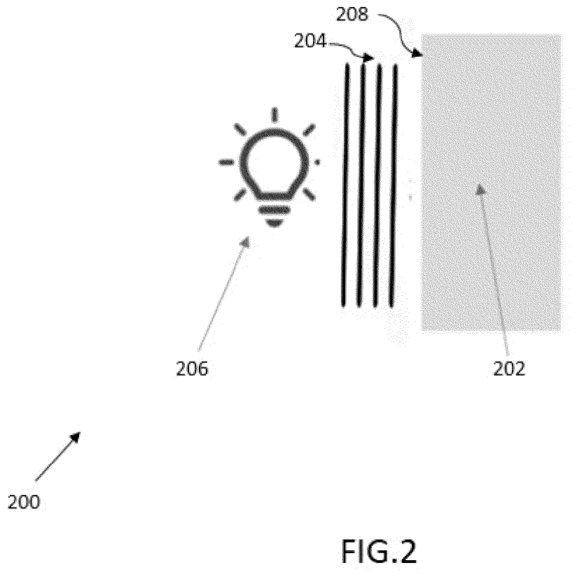

FIG. 2 illustrates an air disinfection system 200 for sterilising air, in accordance with an embodiment of the present invention.

The air disinfection system 200 includes a NiTi based component 202, air 204 in contact with the surface of the NiTi based component 202, and a UVC source 206 for projecting UVC light through the air 204 and onto the NiTi based component 202. The top surface of the NiTi based component 202 includes titanium atoms in oxidised form as TiO₂ 208.

The TiO₂ surfaces in air are exposed to UVC to enhance air quality and control pathogens such as Coronavirus. The exposure of the TiO₂ 208 to UV light of wavelength UVC from the UVC source 206 creates radicals in the boundary layer of air 204 near the surface of the NiTi based component 102. The TiO₂ 208 active sites catalyse the formation of radicals which are highly reactive. The radicals react with organic or microbial matter and significantly increase efficacy of disinfection in the air 204 above that achieved by UVC alone.

HVAC is a key tool for controlling air-borne pathogens such as Coronavirus. Further, in air-based systems, the disinfection chamber could be pressurised. This increases the concentration of radicals.

In an embodiment of the present invention, medical devices and other products that use NiTi as a component can also be disinfected/sterilised/cleaned using the same arrangement of UVC lamp, irradiation and immersion in water or air.

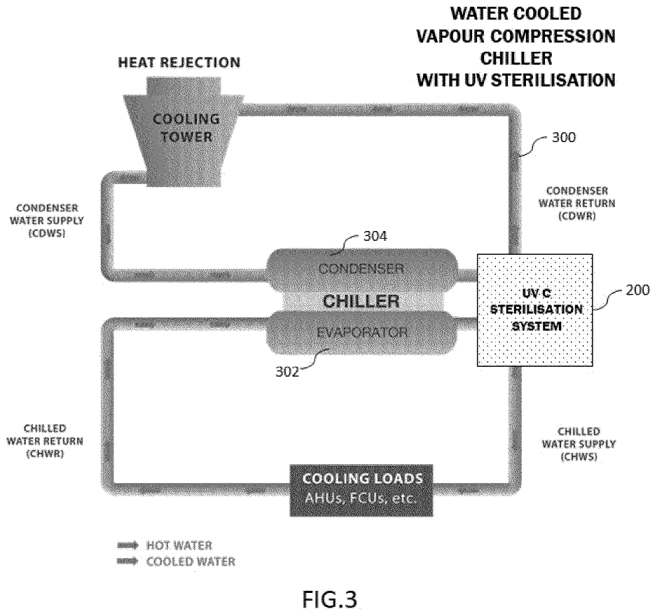

FIG. 3 illustrates integration of the UVC sterilization system 200 in a conventional water-cooled vapour compression chiller 300, in accordance with an embodiment of the present invention. The UVC sterilization system 200 is configured to sterilise cold water outputted from an evaporator 304, and sterilise hot water outputted from a condenser 304, in a manner as described in FIG. 1.

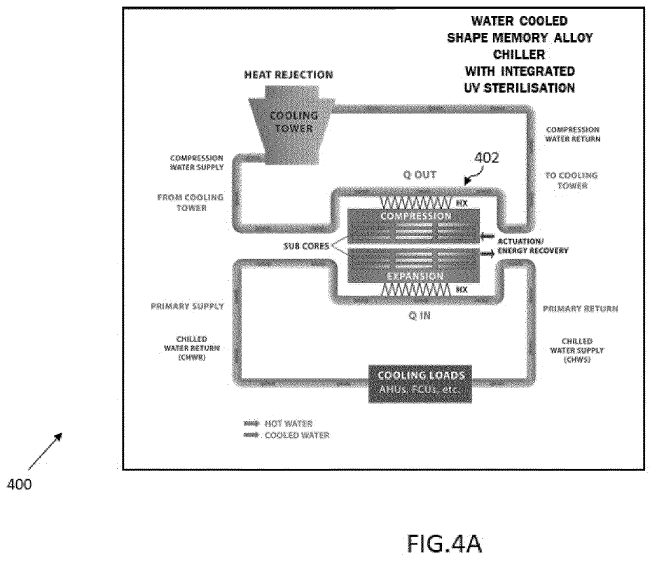

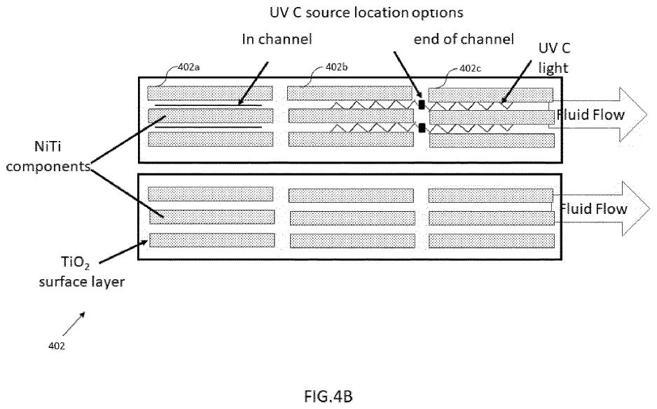

FIG. 4A illustrates a water-cooled SMA chiller 400 with integrated UV sterilization, in accordance with an embodiment of the present invention. The water-cooled SMA chiller 400 is a type of heat pump system.

The water-cooled SMA chiller 400 includes SMA based cores 402 for facilitating heating/cooling of water. An enlarged view of the SMA based core 402 is illustrated with reference to FIG. 4B. As shown, the SMA based core 402 is formed of a plurality of sub-cores 402a, 402b, 402c etc, and each sub-core is made of a NiTi based component. It will be appreciated that water-cooled SMA chiller 400 can be configured in reverse and used in a heating system.

In accordance with an embodiment of the present invention, one or more UVC sources may be provided in channels between adjacent sub-cores 402a, 402b, and 402c. The UVC sources 10a and 10b project UVC light onto the surfaces of the sub-cores 402a and 402b. The UVC light reacts with TiO₂ at the exposed surfaces of the cores 402a, 402b, and 402c, for sterilising water flowing across the SMA core 402, in a manner described in FIG. 1.

In an embodiment of the present invention, the sub cores 402a, 402b and 402c can be designed to increase the reflection of UVC around the heat transfer surfaces.

In an embodiment of the present invention, the cycling between pressurised and depressurised states in the water cooled SMA chiller 402, may increase rate of pathogen destruction during water disinfection. The effectiveness of water disinfection may also depend critically on flow rate, width of flow channel, and any fluid turbulence and recirculation needed for complete dispersal of radicals in the fluid.

In an embodiment of the present invention, sensors may be provided in the water cooled SMA chiller 402 for measuring flow rate, turbulence, etc for increasing the effectiveness of water disinfection. Based on the sensors, factors such as flow rate, turbulence, channel dimensions and recirculation may be controlled and optimised to achieve peak efficacy and efficiency of water disinfection.

In an embodiment of the present invention, the concentration of photocatalytic sites is increased by post-production surface treatment as shown in FIG. 5. One method to achieve this is laser treatment, to cause migration of Ti to the surface and oxidation to TiO₂.

In the specification the terms “comprise, comprises, comprised and comprising” or any variation thereof and the terms include, includes, included and including” or any variation thereof are considered to be totally interchangeable and they should all be afforded the widest possible interpretation and vice versa.

The invention is not limited to the embodiments hereinbefore described but may be varied in both construction and detail.

1. A disinfection system comprising:
a NiTi based component that has an exposed surface including titanium atoms in oxidised form as TiO₂; and
a UVC source for projecting UVC light onto the exposed surface of the NiTi based component to react with the TiO₂ present at the exposed surface, wherein the exposure of the TiO₂ with UVC light creates radicals that denature or break down organic matter present in a fluid between the UVC source and the exposed surface of the NiTi based component.

2. The disinfection system as claimed in claim 1, wherein when the fluid to be sterilised is water, the system further includes a transparent barrier between the UVC source and the NiTi based component to prevent contact of water with the UVC source.

3. The disinfection system as claimed in claim 1 wherein the fluid to be sterilised is air.

4. The disinfection system as claimed in claim 1, wherein the NiTi based component is a part of a medical device which is immersed in a fluid subject to the disinfection system.

5. The disinfection system as claimed in claim 1, wherein the NiTi based component is a Shape Memory Alloy (SMA) element present in a core of a heat pump system that performs cooling and/or heating of water, and the UVC source projects UVC light onto the SMA core to sterilise water as a working fluid flowing through the chamber and over the SMA core.

6. A heat pump system, comprising:
a Shape Memory Alloy (SMA) element having a NiTi based core positioned in a housing and adapted to absorb heat and store energy in response to a fluid inputted at a first temperature;
a UVC source positioned at a pre-defined distance from the housing for projecting UVC light towards an exposed surface of the NiTi based core to react with TiO₂ present at the exposed surface of the NiTi based core, wherein the exposure of the TiO₂ with UVC light creates radicals that denature or break down organic matter present in fluid in the housing; and
a transparent barrier between the UVC source and the NiTi based core to prevent contact of water with the UVC source, when the fluid is water.

7. The heat pump system as claimed in claim 6 further comprising sensors in the heat pump system for measuring flow rate, width of flow channel, and fluid turbulence to enable the efficacy and efficiency of water disinfection to be enhanced.

8. The heat pump system as claimed in claim 6, wherein the cycling between pressurised and depressurised states in the heat pump system increases a rate of pathogen destruction during water disinfection.

9. The heat pump system as claimed in claim 6, wherein the SMA core is designed to increase the reflection of UVC around the exposed surface.

10. The heat pump system as claimed in claim 6, wherein water disinfection prevents build-up of a biofilm on the exposed surface of the SMA core.

11. The heat pump system as claimed in claim 6, wherein the fluid to be sterilised is air.

12. A disinfection method comprising:
providing a NiTi based component that has an exposed surface including titanium atoms in oxidised form as TiO₂; and
projecting UVC light onto the exposed surface of the NiTi based component to react with the TiO₂ present at the exposed surface, wherein the exposure of the TiO₂ with UVC light creates radicals that denature or break down organic matter present in a fluid between the UVC source and the exposed surface of the NiTi based component.
